(19) United States
(12) Patent Application Publication
(10) Pub. No.: US 2007/0128247 A1
(43) Pub. Date: Jun. 7, 2007

(54) Method for Ionically Cross-Linking Polysaccharide Material for Thin Film Applications and Products Produced Therefrom

(76) Inventor: Yasushi Pedro Kato, Pembroke Pines, FL (US)

Correspondence Address: GORDON & JACOBSON, P.C., 60 LONG RIDGE ROAD, SUITE 407, STAMFORD, CT, 06902, US

(21) Appl. No.: 11/565,055

(22) Filed: Nov. 30, 2006

Related U.S. Application Data
(60) Provisional application No. 60/741,515, filed on Dec. 1, 2005.

(51) Int. Cl. A61L 15/00

(52) U.S. Cl. 424/423; 424/445; 427/2.24

(57) ABSTRACT

A method for producing ionically cross-linked polysaccharide material (e.g. pectin) that includes dissolving the material in a first liquid solution that includes a dissolving liquid. The first liquid solution is applied to a workpiece to form a polysaccharide-based coating on the workpiece. The coating is dried to remove a substantial portion of the dissolving liquid. Subsequent to drying, the coating is exposed to a second liquid solution that includes a compound that promotes ionic cross-linking of the polysaccharide-based coating. In the preferred embodiment, the dissolving liquid comprises water and possibly a polar solvent. The ionic cross-linking compound preferably comprises a divalent cation such as calcium (Ca²⁺), or possibly strontium (Sr²⁺), magnesium (Mg²⁺), barium (Ba²⁺), or other multivalent ions. Such a method forms a uniform ionically cross-linked film and/or coating for diverse applications, including medical devices such as implantable vascular grafts, stent-grafts and/or stents.

This application claims the benefit of provisional application 60/741,515 filed on Dec. 1, 2005 which is hereby incorporated by reference herein in its entirety.

### BACKGROUND OF THE INVENTION,

1. Field of the Invention

This invention relates to methods for cross-linking polysaccharide materials, in particular low methoxy pectin and products produced from these materials.

2. State of the Art

Polysaccharides are polymers composed of complex chains of carbohydrate groups. This complexity arises due to the presence of glycosidic links between the carbohydrate groups that allow for branching of polymer structures. Because of the molecular complexity, polysaccharides are generally insoluble and amorphous. The class of polysaccharides includes substances such as cellulose, glycogen, pectin, and starch.

Pectins are available as powders. Typically, pectin is dissolved in water and mixed to produce a 0.1-20% solids content solution. The viscosity of the solution increases rapidly with solids content and becomes quite thick at 5% solids content. This dissolving process can be aided by using hot water (e.g., water at 80° C.). Unlike crystal powders, pectin powders tend to clump as they first swell before dissolving into solution. Pectins are generally not soluble in polar solvents such as alcohols. Therefore, the addition of such polar solvent(s) aid in dispersing the pectin powder. In the food industry, sugar is typically added to pectins to facilitate dissolving and to prevent clumping by keeping the pectin grains apart.

Typically, a pectin solution remains in solution form until a gelling agent is introduced. For low methoxy (LM) pectins, calcium (Ca²⁺) ions are typically added to the solution for gelling. These ions require a minimum concentration in order to yield gels with desired properties. Excessive concentrations cause pre-gelation and a tendency for syneresis to occur. Syneresis is the process of moisture expulsion (or removal) as the gel shrinks or conformation changes. The calcium reactivity of a specific LM pectin depends upon the degree of esterification of the specific pectin and the uniformity among molecules of the lot. When Ca²⁺ ions are added to the pectin solution for gelling, the solution immediately starts to gel and thicken. This impedes or complicates the formation of a uniform pectin coating especially for films.

Pectin-based coatings have been proposed for medical devices including implantable, percutaneous, transcutaneous, or surface applied medical devices, such as vascular stents, stent-grafts, grafts, catheters, bone screws, joint repair implants, tissue repair implants, feed tubes, shunts, endotracheal tubes, etc. See U.S. Patent Pub. 2003/0158958, U.S. Patent Pub. 2003/004559 and U.S. Pat. No. 6,723,350. A stent is a generally longitudinal tubular device formed of biocompatible material, preferably a metallic or plastic material. Stents are useful in the treatment of stenosis and strictures in body vessels, such as blood vessels. It is well known to employ a stent for the treatment of diseases of various body vessels. The device is implanted either as a “permanent stent” within the vessel to reinforce collapsing, partially occluded, weakened, or abnormally dilated sections of the vessel or as a “temporary stent” for providing therapeutic treatment to the diseased vessel. Stents are typically employed after angioplasty of a blood vessel to prevent restenosis of the diseased vessel. Stents may be useful in other body vessels, such as the urinary tract and the bile duct. A stent-graft employs a stent inside or outside a graft. The graft is generally a longitudinal tubular device formed of biocompatible material, typically a woven polymeric material such as Dacron or polytetrafluroethylene (PTFE). Stent-grafts are typically used to treat aneurysms in the vascular system. Bifurcated stent-grafts are used to treat Abdominal Aortic Aneurysms. Grafts are typically impermeable to the body fluid (e.g., blood in vascular grafts) that flows through the graft such that the body fluid does not leak out through its wall(s).

Stents, stent-grafts, and grafts typically have a flexible configuration that allows these devices to be configured in a radially compressed state for intraluminal catheter insertion into an appropriate site. Once properly positioned, the devices radially expand such that they are supported within the body vessel. Radial expansion of these devices may be accomplished by an inflatable balloon attached to a catheter, or these devices may be of the self-expanding type that will radially expand once deployed.

U.S. Patent Pub. No. 2003/0158598 describes the coating of stents, stent-grafts, and grafts with a drug-loaded polymer matrix and pectin. The pectin degrades over time and is used to control the release rate of the drug loaded into the polymer matrix. U.S. Patent Pub. No. 2003/0004559 describes a vascular graft employing inner and outer microporous expanded polytetrafluoroethylene (ePTFE) tubes that are formed in separate extrusion processes. An intermediate elastomeric layer is disposed between the two tubes. The intermediate layer may be impregnated with a pectin gel to provide enhanced sealing capabilities.

U.S. Pat. No. 6,723,350 describes a lubricious coating applied to a wide variety of medical devices. The coating can be realized from pectin-based compound prepared from a liquid medium having a gel-like consistency.

In each of these applications, the prior art methodology for applying the polysaccharide-based film to the respective device impedes or complicates the formation of a uniform coating as described above. Thus, there remains a need in the art to provide an improved method for the formation of polysaccharide-based films and coatings that are suitable for applications requiring uniform coatings, including implantable medical devices such as stents, stent-grafts, and grafts.

### SUMMARY OF THE INVENTION

It is therefore an object of the invention to provide a method for ionically cross-linking polysaccharide material that is suitable for applications requiring uniform coatings or films.

It is a further object of the invention to provide a medical device such as a stent, stent-graft, and/or graft that has a uniform coating(s) of ionically cross-linked polysaccharide material, such coatings that are biocompatible and thus suitable for implantation into the human body.

It is another object of the invention to provide such coatings that are suitable for use in vascular devices including stents, stent-grafts, and grafts.

It is yet another object of the invention to provide a vascular graft that employs a uniform coating of ionically cross-linked polysaccharide material to seal the vascular graft such that blood does not leak through its wall(s).

It is a further object of the invention to provide a method for ionically cross-linking polysaccharide material with a gradient such that the outer surface of the material has a higher cross-linking density than the inner surface of the material.

In accord with these objects, an ionically cross-linked polysaccharide-based film is provided that is suitable for medical implant applications. Cross-linked polysaccharide films have good flexibility characteristics and can provide distinctly uniform coatings impermeable to blood that seal medical implant devices. These films are smooth in appearance and are particularly suited for use with stents, stent-grafts, and vascular grafts.

According to a first, preferred embodiment, a method for producing ionically cross-linked polysaccharide material is provided whereby a polysaccharide (e.g. pectin) is first dissolved into a solution and applied to a medical implant device. The polysaccharide solution is then dried to form a coating and then cross-linked with an initiator.

According to a second embodiment, a medical device is described that incorporates an ionically cross-linked polysaccharide.

According to yet another embodiment, a medical device is described that includes an ionically cross-inked pectin polysaccharide in a multi-layer structure of films.

Additional objects and advantages of the invention will become apparent to those skilled in the art upon reference to the detailed description taken in conjunction with the provided figure.

### BRIEF DESCRIPTION OF THE DRAWINGS

FIG. 1 is a schematic diagram of a vascular graft formed with an ionically cross-linked polysaccharide coating in accordance with the present invention.

### DETAILED DESCRIPTION OF THE PREFERRED EMBODIMENTS

For the purposes of this patent application, “ionic cross-linking” refers to a process wherein a polymer (e.g., polysaccharide) is transformed by the formation of ionic bonds between chains of the polymer. The ionic bonds require multivalent counter-ions that form bridges between polymeric chains. A polymer is “ionically cross-linked” after it has been subjected to such ionic cross-linking. A “film” is a layer of material that is no larger than 1 millimeter (mm) in thickness.

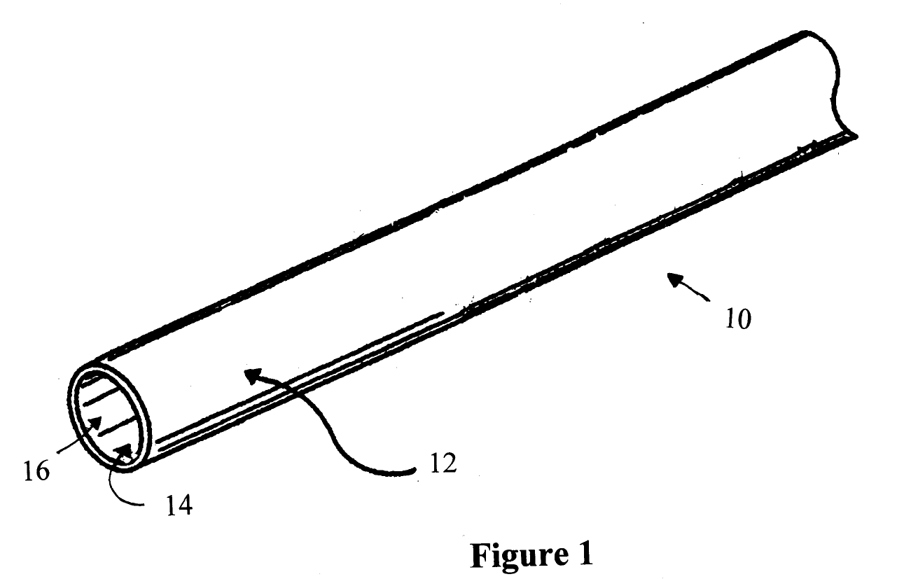

FIG. 1 is a schematic diagram of a vascular graft formed with an ionically cross-linked polysaccharide-based coating in accordance with the present invention.

In accordance with the present invention, a film or coating of ionically cross-linked polysaccharide is realized as follows.

First, a LM pectin powder is dissolved in water to produce a homogenous solution of pectin. This dissolving processing can be aided by using hot water (e.g., water at 80° C.). One or more polar solvent(s) may be added to the solution to aid in dispersing the LM pectin therein. The concentration of LM pectin in the solution can vary between 0.1 to 20% as desired. The LM pectin solution is coated, sprayed, or impregnated onto a workpiece and dried to remove water and any solvents, which produces a dried film of LM pectin on the workpiece. Such drying can be accomplished by subjecting the pectin-coated workpiece to ambient temperatures or to elevated temperatures in a warm oven. Thicker films or coatings of LM pectin can be produced by applying/drying additional LM pectin layers on top of the base layer or by using a higher solids content pectin solution. The dried film of LM pectin may have some retained solvents (for example, between 0 to 20% of the water and solvents may be left behind in the film). The dried film of LM pectin may be removed from the workpiece, if desired.

A liquid solution of calcium chloride in water is prepared. The concentration of calcium chloride can range from near zero to 50% (weight/weight) and preferably between 0.5-10% (weight/weight) and most preferably between 3% and 7% (weight/weight). Other compound(s) can be mixed into the liquid calcium chloride solution as long as the other compound(s) do not compete or steal the calcium ions that are present in the liquid calcium chloride solution. The dried pectin film (and possibly the workpiece if the film was not removed therefrom) is exposed to the liquid calcium chloride solution at a predetermined temperature (e.g., room temperature) for a predetermined time (e.g., 30 minutes). The calcium divalent cations (Ca²⁺ ions) of the liquid solution form bridges between polymeric chains of the LM pectin film submersed therein to thereby ionically cross-link the pectin. The calcium chloride concentration as well as the temperature and time of the exposure to the calcium chloride will affect the degree of the ionic cross-linking up to a point of saturation. Therefore, different degrees of ionic cross-linking can be achieved by varying the calcium chloride concentration as well as the temperature and time of exposure to the calcium chloride solution. These different degrees of ionic cross-linking can provide for different pectin properties as desired. Moreover, as the LM pectin can be built up to a desired thickness by multiple coatings, the calcium chloride concentration and the exposure time can be controlled to produce a gradient of ionically cross-linked layers that have a higher ionically cross-linked density on the outside compared to the inside (inner) layer(s).

One skilled in the art will realize that the ionic cross-linking agent of the bath can comprise other divalent cations such as calcium (Ca²⁺), barium (Ba²⁺), magnesium (Mg²⁺), strontium (Sr²⁺), and/or other multivalent ions. It may also be realized that alternative polysaccharides may be used to produce cross-linked films including cellulose, dextran, gellan gum, and xantham gum.

### EXAMPLE 1

Three pectin solutions were made by dissolving 1, 1.5, and 2 grams of LM pectin powder in 100 milliliters (ml) of distilled water. Ten milliliters of the solution was placed in a weighing dish and allowed to dry at ambient temperature. Ten milliliters of 5% solution of calcium chloride was placed onto the dried pectin film for 30 minutes (mins) at room temperature in order to ionically cross-link the pectin film. The calcium chloride solution was discarded and the ionically cross-linked pectin film was immersed in 10% glycerin in distilled water for 15 minutes at room temperature in order to plasticize the pectin film (otherwise it would be very brittle). The water was evaporated off and the pectin film allowed to dry at room temperature.

### EXAMPLE 2

A 4% pectin solution was made by dissolving LM pectin powder in water. Ten milliliters of the pectin solution was placed in a weighing dish and allowed to dry at 50° C. Ten milliliters of 5% solution of calcium chloride was placed onto the dried film for 30 minutes at room temperature in order to ionically cross-link the pectin film. The calcium chloride solution was discarded and the ionically cross-linked pectin film was immersed in 10% glycerin in distilled water for 15 minutes at room temperature in order to plasticize the pectin film. The pectin film was padded with a paper towel and punched with a #5 punch to make disks. The disks were immersed in 20 milliliters phosphate buffered saline with 5% isopropanol at 37° C. in order to check for pectin dissolution over time. The disks appeared swollen but remained intact for days. When this example was repeated without exposing the pectin films to the 5% calcium chloride solution, the pectin disks dissolved in the phosphate buffered saline in about 60 to 120 minutes without agitation.

### EXAMPLE 3

A 4% pectin solution was made by dissolving LM pectin powder in water. Ten milliliters of the pectin solution was placed in a weighing dish and allowed to dry at 50° C. Ten milliliters of 1% solution of calcium chloride was placed onto the dried film for 30 minutes at room temperature in order to ionically cross-link the pectin film. The calcium chloride solution was discarded and the ionically-cross-linked pectin film was immersed in 10% glycerin in distilled water for 15 minutes at room temperature in order to plasticize the pectin film. The pectin film was padded with a paper towel and punched with a #5 punch to make disks. The disks were immersed in 20 milliliters phosphate buffered saline with 5% isopropanol at 37° C. in order to check for pectin dissolution over time. The disks appeared more swollen than those of Example 2, but remained intact for days. After four days, these disks appeared to be breaking apart. When this example was repeated without exposing the pectin films to the 1% calcium chloride solution, the pectin disks dissolved in the phosphate buffered saline in about 60-120 minutes without agitation.

### EXAMPLE 4

Referring to FIG. 1, a vascular graft 10 of the present invention is shown, including an elongate tubular structure 12 formed of woven fabric mesh. A central lumen 14 extends through the graft 10. The central lumen 14 is defined by the inner wall surface 16 of the tubular structure 12. The central lumen 14 permits the passage of blood through the graft 10 once the graft 10 is properly implanted in the vascular system.

In accordance with the present invention, a uniform ionically cross-linked LM pectin coating was applied to the tubular structure 12. The pectin coating renders the tubular structure 12 impermeable to blood flowing through the central lumen 14. The LM pectin coating will degrade with time in the body by the action of inflammatory cells and host tissue will take its course of healing from inflammation, proliferative to remodeling phases. In the inflammatory phase (which usually takes a few days), platelet aggregation and thrombin will coat the surface and macrophages will start to degrade the LM pectin coating by phagocytosis and possibly enzymatic and oxidative degradation. In the proliferative phase and the final remodeling phase (which usually lasts a few days to a few weeks/months), extracellular matrix and collagen will be formed by fibroblasts onto the interstices of the tubular structure, thereby providing a replacement blood-impermeable layer as a substitute for the pectin layer. The ionically cross-linked LM pectin coating was applied to the tubular structure 12 as follows.

A 4% pectin-20% glycerin solution was made by dissolved LM pectin powder and glycerin in water. The solution was impregnated into the tubular structure 12 and allowed to dry at about 45° C. for 30 minutes. The impregnation was repeated and dried. The coated structure 12 was then immersed in a bath of 5% calcium chloride for 30 minutes at room temperature (e.g., 22° C. to 26° C.) in order to ionically cross-link the pectin coating impregnated on the structure 12. The pectin-coated structure 12 is removed from the calcium chloride bath, rinsed and immersed in distilled water for 15 minutes at room temperature. The distilled water was then discarded and the pectin-coated structure 12 was immersed in 40% glycerin in distilled water for 30 minutes at room temperature in order to plasticize the pectin coating. Finally, the pectin-coated structure 12 was dried at about 45° C. for 30 minutes. The pectin-coated structure 12 was cut in half. One half was immersed in 20 ml phosphate buffered saline with 5% isopropanol at 37° C. in order to check for pectin dissolution over time. The other half was kept as a control sample. A permeability tester was used to test water permeability at 120 mmHg of the two halves. Water permeability was less than 1 ml/(min*cm²) for both halves of the pectin-coated structure 12, which indicates that the pectin coating remained on the structure 12 and did not dissolve. In the preferred embodiment, the uniform ionically cross-linked pectin coating that is applied to the tubular structure 12 is a layer of material that is no larger than 1 millimeter in thickness.

In yet other embodiments of this invention, a uniform ionically cross-linked polysaccharide (e.g. pectin) coating can be applied to other medical devices, such as implantable stents, stent-grafts, and other vascular grafts. In these applications, a polysaccharide solution is coated, sprayed or impregnated onto the respective device and dried to remove water and any solvents, which produces a dried film of polysaccharide on the device. The polysaccharide material can be built up to a desired thickness by multiple coatings/drying steps or by using a higher solids content pectin solution as described above. The polysaccharide-coated device is then immersed (or otherwise subjected) to a bath of calcium chloride (or other suitable ionic cross-linking agent as described above) in order to ionically cross-link the polysaccharide coating. The polysaccharide-coated device is then preferably rinsed, immersed in distilled water, and immersed in glycerin in order to plasticize the polysaccharide coating. Finally, the polysaccharide-coated device is dried. The uniform ionically cross-linked polysaccharide coating can be used to render surfaces of the device impermeable to bodily fluid (e.g., blood in vascular applications) or possibly for controlling the release rate of therapeutic drugs loaded into a release structure (e.g., polymer matrix) disposed under the polysaccharide coating.

The polysaccharide coatings/films described herein have improved uniformity. More particularly, when viewed by a scanning electron microscope (SEM), the polysaccharide coatings/films appear smooth like a sheet. On a textured vascular graft, the film enveloped the textile forming a film as if it was wrapped in cellophane.

The polysaccharide coatings/films described herein can also be used as a lubricious coating layer for a wide variety of medical devices, including catheters, bone screws, joint repair implants, tissue repair implants, feed tubes, shunts, endotracheal tubes, etc. The polysaccharide coatings/films can also be applied to a medical device and used to hold a therapeutic drug for drug delivery purposes. The drug can be mixed with the liquid polysaccharide solution and subsequently applied to part of the medical device, where it is dried and then subjected to a cross-linking agent(s). The drug must not react with the pectin nor with the cross-linking agent(s) to form other entities. In this application, the drug can be eluted from the polysaccharide coating/film as the polysaccharide coating/film slowly degrades over time.

There have been described and illustrated herein several embodiments of a method for forming a uniform ionically cross-linked polysaccharide film or coating and products based thereon. While particular embodiments of the invention have been described, it is not intended that the invention be limited thereto, as it is intended that the invention be as broad in scope as the art will allow and that the specification be read likewise. Thus, while particular concentrations, temperatures, and heating times have been disclosed, it will be appreciated that other such parameters can be used as well. In addition, while applications for particular types of implantable medical devices have been disclosed, it will be understood that the principles of the present invention can be used for other implantable medical devices. Furthermore, while the applications described above utilize polysaccharide-based films and coatings for fluid impermeability and release rate control, it will be understood that other polysaccharide-based films and coating can be used for other applications. It will therefore be appreciated by those skilled in the art that yet other modifications could be made to the provided invention without deviating from its spirit and scope as claimed.

What is claimed is:

1. A method for producing a polysaccharide-based coating comprising:
a) dissolving a polysaccharide material in a first liquid solution that includes a dissolving liquid;
b) applying the first liquid solution to a workpiece to form a polysaccharide-based coating on the workpiece;
c) drying the polysaccharide-based coating to remove a substantial portion of the dissolving liquid; and
d) exposing the polysaccharide-based coating to a second liquid solution subsequent to drying, the second liquid solution including a compound that provides ionic cross-linking of the polysaccharide-based coating.

2. The method according to claim 1, wherein:
said polysaccharide material is pectin.

3. The method according to claim 2, wherein:
said dissolving liquid includes water.

4. The method according to claim 2, wherein:
the first liquid solution includes a polar solvent.

5. The method according to claim 2, wherein:
the compound that provides ionic cross-linking of the polysaccharide-based coating includes at least one divalent cation.

6. The method according to claim 5, wherein:
the at least one divalent cation includes Ca²⁺.

7. The method according to claim 6, wherein:
the second liquid solution includes calcium chloride.

8. The method according to claim 6, wherein:
the second liquid solution includes a solution of calcium chloride and water with a concentration of calcium chloride by weight in a range between 3% and 7%.

9. The method according to claim 5, wherein:
the at least one divalent cation is selected from the group consisting of Sr²⁺, Mg²⁺, and Ba²⁺.

10. The method according to claim 2, wherein:
the compound that provides ionic cross-linking of the polysaccharide-based coating includes at least one multivalent ion.

11. The method according to claim 2, further comprising:
after the drying step and before the exposing step, reapplying the first liquid solution to the workpiece and drying the resultant structure to realize a multi-layer polysaccharide-based coating.

12. The method according to claim 11, further comprising:
controlling the exposing step to provide a gradient of the density of ionically cross-linked polysaccharide material from an outer portion to an inner portion of the ionically cross-linked polysaccharide material.

13. A method of manufacturing an implantable medical device comprising:
a) providing at least one implantable part;
b) dissolving a polysaccharide material in a first liquid solution that includes a dissolving liquid;
c) applying the first liquid solution to the at least one implantable part to form a polysaccharide-based coating on the at least one implantable part;
d) drying the polysaccharide-based coating to remove a substantial portion of the polysaccharide-dissolving liquid; and
e) subsequent to drying, exposing the polysaccharide-based coating to a second liquid solution, the second liquid solution including a compound that promotes ionic cross-linking of the polysaccharide-based coating.

14. The method of claim 13, wherein:
the polysaccharide material and the polysaccharide-based coating are pectin.

15. The method according to claim 14, wherein:
the dissolving liquid includes water.

16. The method according to claim 14, wherein:
the first liquid solution includes a polar solvent.

17. The method according to claim 14, wherein:
the compound that provides ionic cross-linking of the polysaccharide-based coating includes at least one divalent cation.

18. The method according to claim 17, wherein:
the divalent cation includes Ca²⁺.

19. The method according to claim 18, wherein:
the second liquid solution includes calcium chloride.

20. The method according to claim 19, wherein:
the second liquid solution includes a solution of calcium chloride and water with a concentration of calcium chloride by weight in a range between 3% and 7%.

21. The method according to claim 17, wherein:
the divalent cation is selected from the group consisting of Sr²⁺, Mg²⁺, and Ba²⁺.

22. The method according to claim 14, wherein:
the compound that provides ionic cross-linking of the polysaccharide-based coating comprises a multivalent ion.

23. The method according to claim 14, further comprising:
after the drying of d) and before the exposing of e), reapplying the first liquid solution to the at least one implantable part and drying the resultant structure to realize a multi-layer polysaccharide-based coating.

24. The method according to claim 23, further comprising:
controlling the exposing step to provide a gradient of the density of ionically cross-linked polysaccharide material from an outer portion to an inner portion of the ionically cross-linked polysaccharide material.

25. The method according to claim 14, wherein:
the at least one implantable part includes a tubular portion of a vascular graft.

26. The method according to claim 25, wherein:
the tubular portion is realized from a woven fabric that is sealed by the polysaccharide-based coating such that blood does not leak through its annular wall.

27. The method according to claim 14, wherein:
the at least one implantable part includes a portion of a stent.

28. The method according to claim 14, wherein:
the at least one implantable part includes a portion of a stent-graft.

29. A medical device comprising:
at least one film of a material that includes an ionically cross-linked polysaccharide, the at least one film being characterized by at least one of the following:
(i) the at least one film being a part of a multi-layer structure comprising a plurality of film layers disposed on top of one another, each film layer including ionically cross-linked polysaccharide;
(ii) the at least one film having a thickness of less than 1 millimeter; and
(iii) the at least one film provides a uniform coating.

30. The medical device according to claim 29, wherein:
a density of the ionically cross-linked polysaccharide changes between film layers of the multi-layer structure.

31. The medical device according to claim 29, wherein:
the medical device is selected from the group consisting of stents, stent-grafts, and vascular grafts.

32. The medical device according to claim 29, wherein:
the at least one film of a material is smooth in appearance.

33. The medical device according to claim 29, wherein:
the ionically cross-linked polysaccharide is pectin.
